(19) United States
(12) Patent Application Publication
(10) Pub. No.: US 2024/0054878 A1
(43) Pub. Date: Feb. 15, 2024

(54) NOTIFICATION OPTIMIZATION SYSTEM AND SYSTEM MANAGEMENT SERVER

(71) Applicant: PARAMOUNT BED CO., LTD., Tokyo (JP)

(72) Inventors: Junko NISHIMURA, Koto-ku Tokyo (JP); Sota NISHIURA, Koto-ku Tokyo (JP); Masato SHIMOKAWA, Koto-ku Tokyo (JP)

(21) Appl. No.: 18/178,453

(22) Filed: Mar. 3, 2023

(30) Foreign Application Priority Data
Aug. 12, 2022 (JP) 2022-128987

(51) Int. Cl. G08B 21/18; G06F 3/0482; G06F 3/04847

(52) U.S. Cl. CPC G08B 21/18; G06F 3/0482; G06F 3/04847

(57) ABSTRACT

The notification optimization system includes: a first setting unit to which a first notification setting in a first time zone including noon is set, the first notification setting being selected from a plurality of notification settings; a second setting unit to which a second notification setting in a second time zone including twelve midnight is set, the second notification setting being selected from the plurality of notification settings; and a controller configured to perform a notification based on settings of the first setting unit or the second setting unit.

### CROSS-REFERENCE TO RELATED APPLICATIONS

This application is based upon and claims the benefit of priority from the prior Japanese Patent Application No. 2022-128987, filed on Aug. 12, 2022; the entire contents of which are incorporated herein by reference.

### FIELD

One or more embodiments relate to a notification optimization system and a system management server.

### BACKGROUND

A system is known in which a state of a subject is determined using a sensor installed on a bed, and a determination result thereof is displayed.

In a case where a state of a user using a bed provided in a facility such as a hospital or an elderly care facility changes, for example, notification of a changed content is performed based on individual settings for the users.

However, in recent years, there is a significant change in an environment where a service is provided in a facility. For example, the number of users using a facility is greatly increased, and an amount of contents set for management accompanying changes in the provided service are greatly increased. In addition to these, contents necessary for notification differ depending on a state of a user and a time window. For example, since the number of staff members of the facility is different for daytime and nighttime, and states of the users are also different, for example, a user is awake or is asleep, contents to be notified change.

Here, it is conceivable to precisely make a setting for each of the users in detail. However, in view of a situation where the number of the users of the facility is increased and the amount of the contents to be set is greatly increased, and a situation where the settings need to be changed according to changes in conditions of the users, the labor of the staff members is greatly increased by precisely making the settings.

In addition to the labor, it is also difficult for a staff member not familiar with the setting to set a notification content in detail. For this reason, it is often the case where the staff member does not know an optimum setting and uses a default setting as it is or where the staff member turns on all notifications. In these cases, the number of notifications increases, which causes a decrease in operational efficiency.

An embodiment provides a notification optimization system and a system management server that are capable of easily optimizing a notification setting.

### SUMMARY

The notification optimization system according to the embodiment includes: a first setting unit to which a first notification setting in a first time zone including noon is set, the first notification setting being selected from a plurality of notification settings; a second setting unit to which a second notification setting in a second time zone including twelve midnight is set, the second notification setting being selected from the plurality of notification settings; and a controller configured to perform a notification based on settings of the first setting unit or the second setting unit.

### BRIEF DESCRIPTION OF THE DRAWINGS

FIG. 1 is a diagram illustrating an example of an entire network configuration according to a first embodiment.

FIG. 2 is a diagram illustrating an example of a network configuration within a facility.

FIG. 3 is a perspective view illustrating an example of an appearance of the motorized bed.

FIG. 4 is a diagram illustrating an example of a control configuration of the motorized bed.

FIG. 5 is a plan view illustrating an example of the detection device.

FIG. 6 is a diagram illustrating an example of a control configuration of the detection device.

FIG. 7 is a diagram illustrating an example of a control configuration of the facility management server.

FIGS. 8A to 8F are diagrams showing an example of the symbols corresponding to the states of the user.

FIGS. 9A to 9F are diagrams showing an example of the setting for the outlines of the symbol display.

FIG. 10 is a diagram illustrating an example of the user information stored in the user information memory.

FIG. 11 is a diagram illustrating an example of a notification setting of the notification setting unit.

FIG. 12 is a diagram illustrating an example of a preset setting of “falling-down prevention”.

FIG. 13 is a flowchart illustrating an example of the state display program.

FIG. 14 is a diagram illustrating a display example of the states of the users displayed on the display.

FIG. 15 is a diagram illustrating an example of a pop-up screen.

FIG. 16 is a diagram illustrating an example of the detail screen.

FIG. 17 is a diagram illustrating an example of a control configuration of the system management server.

FIG. 18 is a diagram illustrating an example of a dashboard screen.

FIG. 19 is a diagram illustrating an example of a dashboard screen.

FIG. 20 is a diagram illustrating an example of a pop-up display.

FIG. 21 is a diagram illustrating an example of a dashboard screen.

FIG. 22 is a diagram illustrating an example of a pop-up screen.

FIG. 23 is a diagram illustrating an example of a display range setting screen according to a second embodiment.

FIG. 24 is a diagram illustrating an example of a display range selection screen.

FIG. 25 is a diagram illustrating an example of the entire network configuration according to a third embodiment.

### DETAILED DESCRIPTION

Hereinafter, one or more embodiments are described with reference to the drawings.

The drawings are schematic or conceptual, and the relations between the thickness and width of portions, the proportions of sizes among the portions and the like are not necessarily the same as the actual values thereof. Further, the dimensions and proportions may be illustrated differently among the drawings, even for identical portions.

In the description and the drawings, components similar to those described previously with reference to earlier figures are marked with the same reference numerals, and the detailed description thereof is omitted as appropriate.

### First Embodiment

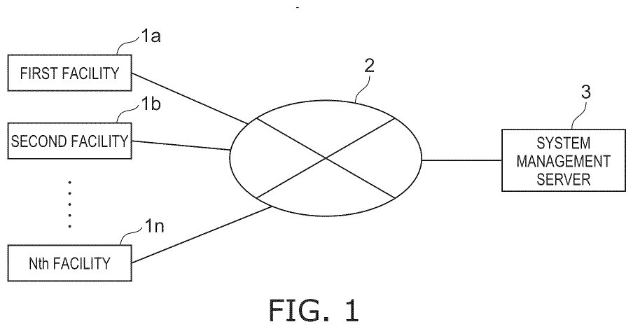

FIG. 1 is a diagram illustrating an example of an entire network configuration.

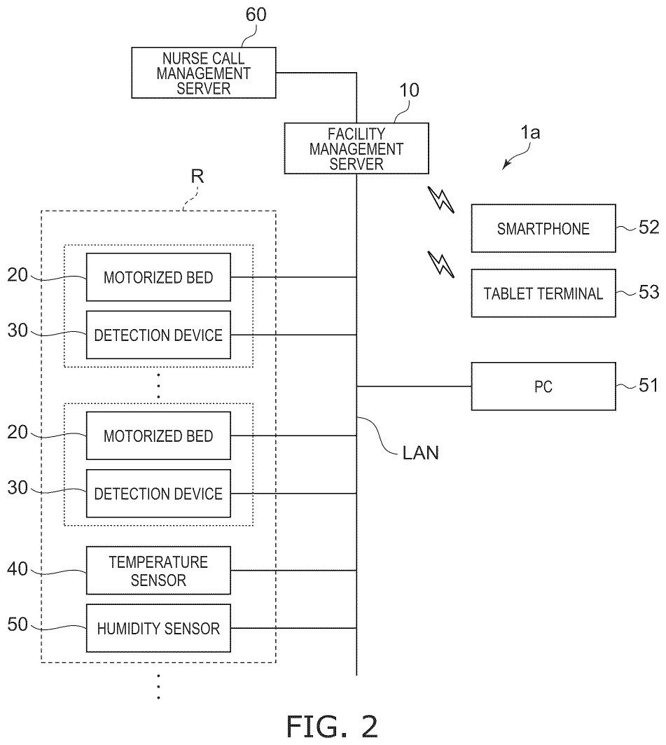

As illustrated in FIG. 1, a first facility 1a, a second facility 1b, . . . , and an Nth facility 1n are communicably connected to a system management server 3 via a network 2. The network 2 is, for example, the Internet. Networks within the first facility 1a, the second facility 1b, . . . , and the Nth facility 1n will be described later with reference to FIG. 2. The first facility 1a, the second facility 1b, . . . , and the Nth facility 1n are, for example, hospitals and elderly care facilities. The system management server 3 is constructed, for example, on a cloud. The system management server 3 manages user information on users using the first facility 1a, the second facility 1b, . . . , and the Nth facility 1n for each facility. FIG. 1 illustrates a case where there are a plurality of facilities, and the number of facilities may be one.

FIG. 2 is a diagram illustrating an example of a network configuration within a facility. In the present embodiment, the network constructed within the first facility 1a will be described as an example.

As illustrated in FIG. 2, the network within the first facility 1a includes a facility management server 10 that is a notification optimization system, a plurality of sets of a motorized bed 20 and a detection device 30, a temperature sensor 40, a humidity sensor 50, and information processors each including a display, for example, a personal computer (PC) 51, a smartphone 52, and a tablet terminal 53. The facility management server 10 is communicably connected to the motorized beds 20, the detection devices 30, the temperature sensor 40, the humidity sensor 50, the personal computer (PC) 51, the smartphone 52, and the tablet terminal 53. In the present embodiment, the detection device 30 is disposed on the motorized bed 20, and instead of the motorized bed 20, the detection device 30 may be disposed on a general bed that does not perform an operation. The facility management server 10 is communicably connected to a nurse call management server 60. When a nurse call button of an operation device 26 to be described later is pressed, the nurse call management server 60 receives information indicating that a nurse call occurs and identifying the motorized bed 20 where the nurse call occurs, and transmits the received information to the facility management server 10. The notification of the nurse call may be performed from the operation device 26 to be described later to the facility management server 10 without through the nurse call management server 60.

The network within the first facility 1a is, for example, a local area network (LAN). In FIG. 2, a case where the facility management server 10 is connected to the motorized beds 20, the detection devices 30, and the PC 51 in a wired manner, and is connected to the smartphone 52 and the tablet terminal 53 in a wireless manner is described as an example. The present embodiment is not limited thereto, and any communicable connection may be used. For example, the facility management server 10 may be communicably connected to the motorized beds 20 and the detection devices 30 in a wireless manner.

The plurality of sets of the motorized bed 20 and the detection device 30, the temperature sensor 40, and the humidity sensor 50 are disposed, for example, in a room R of the first facility 1a. In FIG. 2, only one room R is shown, and the number of rooms R correspond to the number of rooms of the first facility 1a. The motorized bed 20 is, for example, a medical bed provided in an elderly care facility, a hospital or the like. The detection device 30 detects a state of a user using the motorized bed 20 disposed in the first facility 1a. Details of the motorized bed 20 and the detection device 30 will be described below. In the present embodiment, a case where the detection device 30 is disposed on the motorized bed 20 is described, and the detection device 30 may be disposed on a bed that does not perform an operation and may detect a state of a user using the bed.

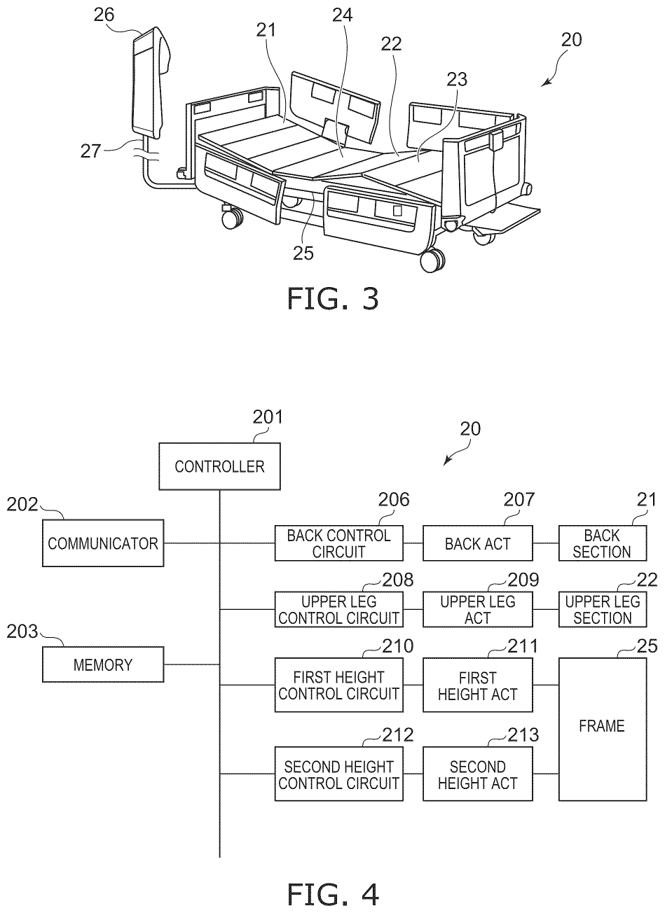

FIG. 3 is a perspective view illustrating an example of an appearance of the motorized bed 20.

As illustrated in FIG. 3, the motorized bed 20 includes a back section 21, an upper leg section 22, a lower leg section 23, a seat section 24, and a frame 25. The back section 21, the upper leg section 22, the lower leg section 23, and the seat section 24 are provided on an upper side of the frame 25. The back section 21 and the upper leg section 22 operate independently. The lower leg section 23 operates in conjunction with an operation of the upper leg section 22. The seat section 24 is fixed onto the frame 25. The frame 25 operates in a height direction.

The operation device 26 is communicably connected to the motorized bed 20 via a communication cable 27. The operation device 26 instructs the motorized bed 20 to operate via the communication cable 27 based on an operation of the user. For example, the nurse call button (not shown) is provided in the operation device 26. The nurse call button is pressed in a case where support by a nurse (or a care staff member) is required, for example, in a case where the user desires a care service. In the present embodiment, the case where the operation device 26 and the motorized bed 20 are connected in a wired manner by using the communication cable 27 is described, and the operation device 26 and the motorized bed 20 may be communicably connected in a wireless manner.

FIG. 4 is a diagram illustrating an example of a control configuration of the motorized bed 20.

As illustrated in FIG. 4, the motorized bed 20 includes a controller 201, a communicator 202, a memory 203, a back control circuit 206, a back actuator (ACT) 207, an upper leg control circuit 208, an upper leg actuator (ACT) 209, a first height control circuit 210, a first height actuator (ACT) 211, a second height control circuit 212, and a second height actuator (ACT) 213.

The controller 201 includes a CPU, a ROM, a RAM and the like. The controller 201 controls an operation of the motorized bed 20 based on an instruction from the operation device 26. The communicator 202 is an interface for transmitting and receiving information to and from the operation device 26. The communicator 202 is an interface for transmitting and receiving information to and from the facility management server 10. The memory 203 stores various programs and various data. The various programs are, for example, programs for operating the back section 21, the upper leg section 22, and the frame 25. For example, the various data store values at which the back section 21, the upper leg section 22, and the frame 25 are operated.

The back control circuit 206 controls the back ACT 207 to operate the back section 21. The back section 21 supports a back to a head part of a user lying on the motorized bed 20, and operates to raise the back of the user. The upper leg control circuit 208 controls the upper leg ACT 209 to operate the upper leg section 22. The upper leg section 22 operates to bend a knee part of the user together with the lower leg section 23. The first height control circuit 210 controls the first height ACT 211 to control a height of the frame 25 on a head part side. The second height control circuit 212 controls the second height ACT 213 to control a height of the frame 25 on a lower leg side. The first height ACT 211 and the second height ACT 213 are actuators for changing the height of the frame 25 of the motorized bed 20. When the first height ACT 211 and the second height ACT 213 change by the same amount in the height direction, the frame 25 operates in the height direction. When a difference between lengths of the first height ACT 211 and the second height ACT 213 is set in the height direction, the frame 25 operates to be inclined with respect to an installation surface of the motorized bed 20. A load sensor is housed in each of the first height ACT 211 and the second height ACT 213. Loads detected by the load sensors are transmitted to the controller 201. The controller 201 can estimate the state of the user using the motorized bed 20 based on the loads detected by the load sensors. For example, the controller 201 can use a body weight of the user, the loads detected by the two load sensors, and a calculated position of center of gravity to estimate the state of the user such as a sitting-up state, an edge sitting position state, a bed-departure state, a wandering state and the like. A state of the motorized bed 20 operated in this way and the state of the user are transmitted, via the communicator 202, to the facility management server 10 in real time in association with a serial number of the motorized bed 20.

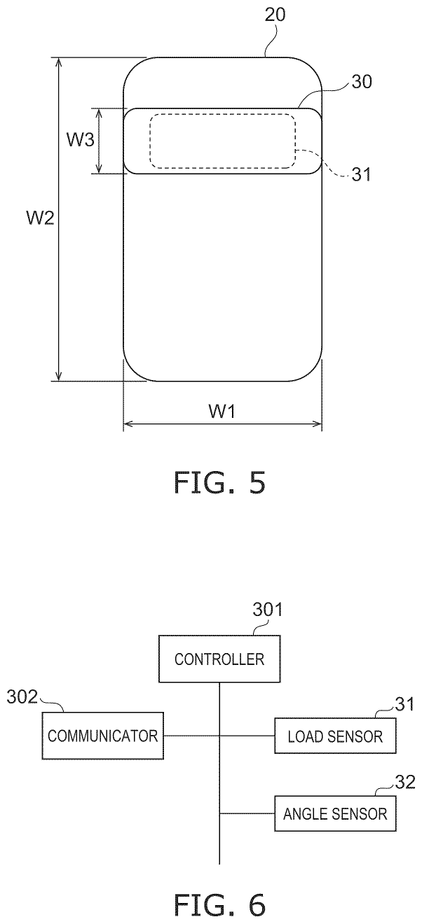

The detection device 30 is disposed on the motorized bed 20. FIG. 5 is a plan view illustrating an example of the detection device 30. In the present embodiment, a case where the detection device 30 is disposed on the motorized bed 20 is described, and the detection device 30 may be disposed on a bed that does not perform an operation.

As illustrated in FIG. 5, for example, the detection device 30 has a length the same as a width W1 of the motorized bed 20 and a predetermined width W3 of a width W2 of the motorized bed 20 in a longitudinal direction. That is, the detection device 30 is formed in a sheet shape having a slight thickness. The detection device 30 is disposed, for example, below a mattress disposed on the motorized bed 20. The detection device 30 is disposed such that the width W3 is located in a manner of including a width of a shoulder part of the user when the user lies on the mattress. A width W1 of the detection device 30 does not coincide with the width W1 of the motorized bed 20, and may be a width that is narrower than the width W1, and may be a width at which the state of the user can be detected by a load sensor 31. The detection device 30 includes the load sensor 31 that measures a load applied to the detection device 30, and an angle sensor (not shown in FIG. 5) therein.

FIG. 6 is a diagram illustrating an example of a control configuration of the detection device 30.

As illustrated in FIG. 6, the detection device 30 includes a controller 301, a communicator 302, the load sensor 31, and an angle sensor 32. In addition to a respiratory rate and a heartbeat rate of a user using the detection device 30, the controller 301 can detect a state of the user such as a sleeping state, a waking-up state, the sitting-up state, and the bed-departure state based on the load detected by the load sensor 31. The angle sensor 32 acquires an angle of the back section 21. The controller 301 transmits information indicating the state of the user detected in this way as user information, via the communicator 302, to the facility management server 10 in real time in association with a serial number for identifying the detection device 30. In the present embodiment, a case where the angle of the back section 21 is acquired by the angle sensor 32 is described, and the angle of the back section 21 may be acquired from the motorized bed 20 by communicating with the motorized bed 20. When the detection device 30 is configured to communicate with the motorized bed 20, the detection device 30 can acquire the state of the motorized bed 20 and the state of the user, and can also transmit information (for example, angle information of the back section 21) acquired from the motorized bed 20 to the facility management server 10 in addition to the information detected by the detection device 30.

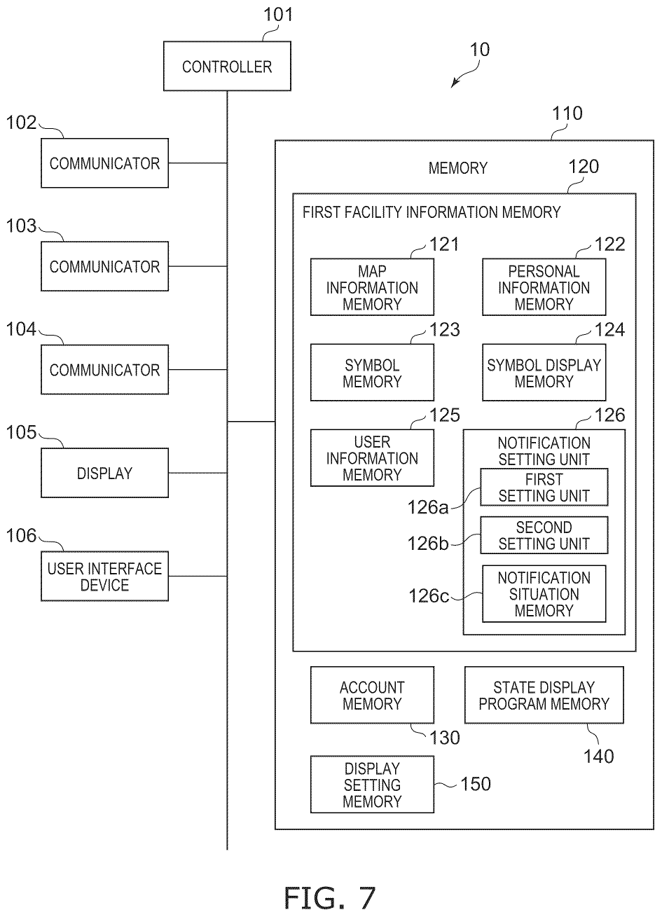

FIG. 7 is a diagram illustrating an example of a control configuration of the facility management server 10.

As illustrated in FIG. 7, the facility management server 10 includes a controller 101, a communicator 102, a communicator 103, a communicator 104, a display 105, a user interface device 106, and a memory 110. The memory 110 includes a first facility information memory 120, an account memory 130, a state display program memory 140, and a display setting memory 150. The first facility information memory 120 includes a map information memory 121, a personal information memory 122, a symbol memory 123, a symbol display memory 124, a user information memory 125, and a notification setting unit 126. The notification setting unit 126 includes a first setting unit 126a, a second setting unit 126b, and a notification situation memory 126c.

The controller 101 controls the components in the facility management server 10. The communicator 102 is an interface that transmits and receives information to and from the system management server 3 via the network 2. The communicator 103 is an interface that transmits and receives information to and from the PC 51. The communicator 104 is an interface for transmitting and receiving information to and from the smartphone 52 and the tablet terminal 53. The display 105 is, for example, a liquid crystal display. For example, the display 105 displays information to a staff member of the first facility 1a via the PC 51, the smartphone 52, and the tablet terminal 53. In the present embodiment, as described later, a user state of a user using the first facility 1a is displayed in real time. The user interface device 106 is a keyboard, a mouse, or the like.

The first facility information memory 120 stores information on the first facility 1a and information on the user using the first facility 1a. That is, the first facility information memory 120 mainly stores information specific to the first facility 1a. The map information memory 121, the personal information memory 122, the symbol memory 123, the symbol display memory 124, the user information memory 125, and the notification setting unit 126 in the first facility information memory 120 will be described.

The map information memory 121 stores map information of the first facility 1a. Here, in the present embodiment, the map information includes a map of, for example, rooms for each floor in the first facility 1a, and positions of the detection devices 30 disposed in the rooms.

The personal information memory 122 stores personal information of the user. For example, the personal information is information for managing, for each identification information of the detection device 30 (the serial number of the detection device) used by the user, care record information, sleep information, information on vital signs (health state) of the user using the detection device 30, and information on environment (temperature, humidity, and the like) in the room acquired by the sensors. Therefore, the personal information includes the serial number of the detection device 30 in addition to contents including a name, an age, a health state, the care record, and the like of the user. In the present embodiment, the association with the map information is performed based on the serial number of the detection device 30. The personal information memory 122 may further include an area where the staff member can input, for example, a special note (a note related to the state or a symptom of the user) as an additional record. It is also possible to use a so-called electronic medical record or the care record as the personal information. In addition, for each room (room number), the user, the serial number of the detection device 30, and identification numbers of the other sensors may be managed in association with each other. In other words, it is sufficient if history information indicating which detection device 30 is used by which user for which time period, and measurement information measured by the sensors can be stored in association with each other.

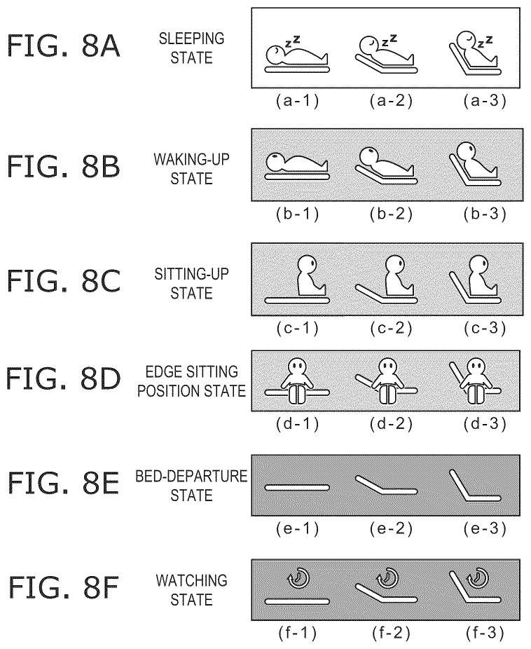

The symbol memory 123 stores symbols corresponding to the states of the user. Here, FIG. 8A to 8F illustrate examples of the symbols corresponding to the states of the user. In the present embodiment, in addition to the symbols indicating the states of the user, the angle of the back section 21 of the motorized bed 20 is also displayed. For a case where the back section 21 is in a horizontal state (0 degree to 10 degrees), a case where the back section 21 has a small angle (11 degrees to 30 degrees), and a case where the back section 21 has a large angle (31 degrees to 75 degrees), three symbols indicating the angles of the back section 21 are set, respectively. For example, as illustrated in (a-1), (a-2), and (a-3) of FIG. 8A, three states of the back section 21 indicating the horizontal state, a small angle state, and a large angle state are set, respectively.

As illustrated in FIGS. 8A to 8F, in the present embodiment, symbols are set for six states including the sleeping state (FIG. 8A), the waking-up state (FIG. 8B), the sitting-up state (FIG. 8C), the edge sitting position state (FIG. 8D), the bed-departure state (FIG. 8E), and a watching state (FIG. 8F) of the user. The sleeping state (FIG. 8A) indicates that the user is in the sleeping state. The symbol indicates that the user is asleep. The waking-up state (FIG. 8B) indicates that the user is in the waking-up state where the user is awake. The symbol indicates the state where the user is awake. The sitting-up state (FIG. 8C) indicates a state where the user sits up without leaning against the back section 21. The symbol indicates a state where the user is awake and sits up. The edge sitting position state (FIG. 8D) indicates that the user is in the edge sitting position state where the user sits on a right side or a left side of the motorized bed 20. The symbol indicates a state where the user sits on one side of the motorized bed 20. The bed-departure state (FIG. 8E) indicates the bed-departure state where the user gets off the motorized bed 20. The symbol indicates a state where the user is not in the motorized bed 20. The watching state (FIG. 8F) indicates a watching state, that is, a state where the bed-departure state continues for a certain period of time. The symbol indicates that the user is not in the motorized bed 20 and also indicates an elapsed time.

In the present embodiment, a background color of the sleeping state (FIG. 8A) is set to a cold color (for example, blue), background colors of the waking-up state (FIG. 8B), the sitting-up state (FIG. 8C), and the edge sitting position state (FIG. 8D) are set to a warm color (for example, orange), and background colors of the bed-departure state (FIG. 8E) and the watching state (FIG. 8F) are set to a gray color. In this way, by setting the background colors differently, the staff member of the facility can visually recognize the states of the user easily.

The symbol display memory 124 stores outlines of a symbol display including the symbols described with reference to FIGS. 8A to 8F. In the present embodiment, regarding the outlines of the symbol display, the outline for the sleeping state is set to a circular shape (an infinite number of angles), and the outline is changed such that the number of angles of the symbol display is reduced as the state changes from the sleeping state to a state where it is necessary to pay attention to the user. That is, the outline of the symbol display in a case where it is most necessary to pay attention is a triangular shape.

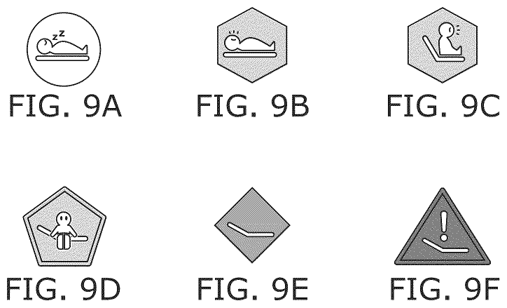

FIGS. 9A to 9F are diagrams showing an example of the setting for the outlines of the symbol display.

As illustrated in FIGS. 9A to 9F, when the user is in the sleeping state, the outline of the symbol display is a circular shape as illustrated in FIG. 9A. As illustrated in FIGS. 9B and 9C, when the user is in the waking-up state and the sitting-up state, the outline of the symbol display is a hexagonal shape. As illustrated in FIG. 9D, when the user is in the edge sitting position state, the outline of the symbol display is a pentagonal shape. In the edge sitting position state illustrated in FIG. 9D, since a display color thereof is the same as that of the state illustrated in FIG. 9C, a further differentiation is achieved by adding a frame. As illustrated in FIG. 9E, when the user is in the bed-departure state, the outline of the symbol display is a rectangular shape. As illustrated in FIG. 9F, in a case where the urgency is high, for example, when there is a nurse call, the outline of the symbol display is a triangular shape. The nurse call is acquired from the nurse call management server 60 in the present embodiment, and the nurse call may be acquired from the operation device 26 of the motorized bed 20 without through the nurse call management server 60. In this way, the number of angles of the symbol display is reduced as the possibility of the user moving around is increased. Since the edge sitting position state illustrated in FIG. 9D is a state close to the waking-up state and the sitting-up state illustrated in FIGS. 9B and 9C, in the case of the edge sitting position state, similarly to the waking-up state and the sitting-up state, the outline of the symbol display may be the hexagonal shape, and the symbol display having the pentagonal shape may not be used.

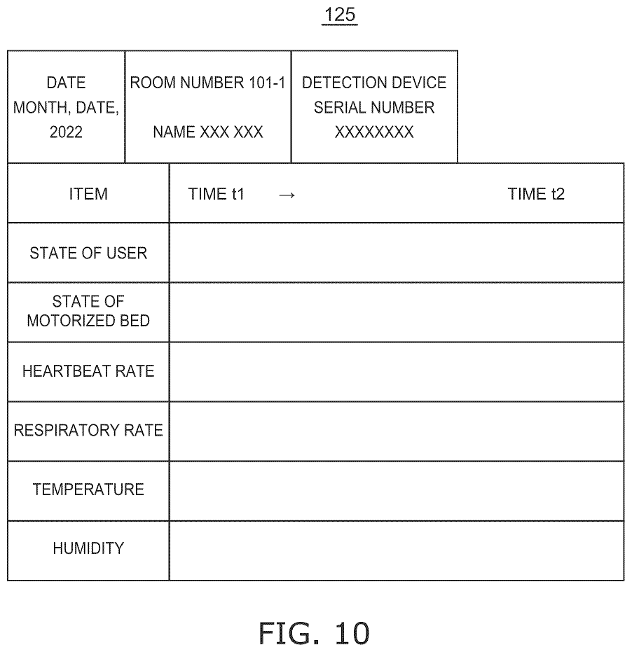

The user information memory 125 sequentially stores the user information transmitted in real time from the detection device 30. FIG. 10 is a diagram illustrating an example of the user information stored in the user information memory 125. The user information memory 125 may also store the information transmitted in real time from the motorized bed 20.

As illustrated in FIG. 10, a date, the name of the user, the room number, the serial number of the detection device 30, the states of the user, the heartbeat rate, the respiratory rate, the temperature, and the humidity are associated with each user, and are displayed on and stored in the user information memory 125 in real time as the time elapses. A time t1 is a time to start a notification process when the user turns into a state set in advance, and a time t2 is a time to end the process for notification. A period from the time t1 to the time t2 is set to a watching time. For example, when the notification process is performed only at night, the time t1 may be 17:00, and the time t2 may be 8:00. In addition, when the time t1 and the time t2 are set to the same time, it is possible to set the process for notification for 24 hours every day. The time t1 and the time t2 may be set to be different for each user.

The account memory 130 stores an account to be described later in detail. Display of the information stored in the memory 110 or the like, information to be registered, an operation of changing the setting and the like are limited according to the account. In the present embodiment, four accounts are stored. The four accounts are an operator account, a facility account, an organization account, and a system management account. The operator account is an account for performing an operation when a user display system according to the present embodiment is introduced into facilities. The facility account is an account for displaying states of users in a facility. The operator account and the facility account are set for each facility. Therefore, in a case of logging in with the operator account and the facility account, display of the states of the users of other facilities and changes in registration information and settings thereof are not available. Meanwhile, when a plurality of facilities are managed, the organization account is, for example, an account allocated to a person of the headquarter who has an administrative authority. Therefore, the organization account can display all states of users of the plurality of managed facilities, and can change registration information and settings thereof. The system management account is an account used by a company that provides the user display system according to the present embodiment. For example, a specific facility account is assigned to the staff member of the first facility 1a. Therefore, the staff member can display the notification settings of the users in the first facility 1a on, for example, the PC 51 by using the facility account. In the notification settings, for example, conditions such as a waking-up detection, a watching detection, and a respiratory rate reduction detection are set, and when the set conditions are detected, a notification is transmitted to the display such as the PC 51 used by the staff member via the facility management server 10.

The notification setting unit 126 performs notification of, for example, the contents set in the facility management server 10 based on the settings set in advance with respect to the states of the users. A first notification setting in a first time zone including noon (12:00 in the daytime) is set to the first setting unit. The first notification setting is selected from a plurality of notification settings. That is, the first time zone is daytime, and in the present embodiment, it is 7:00 to 21:00. A second notification setting in a second time zone including twelve midnight (12:00 at midnight) is set to the second setting unit. The second notification setting is selected from the plurality of notification settings. That is, the second time zone is nighttime, and in the present embodiment, the second time zone is 21:00 to 6:00. Here, the plurality of notification settings include, for example, the settings of the states of the user detected by the detection device 30. The notification situation memory 126c stores a time when a notification is performed, the number of notifications, a content of the notification, and a response situation such as whether the notification is responded. The setting by the notification setting unit 126 will be described later.

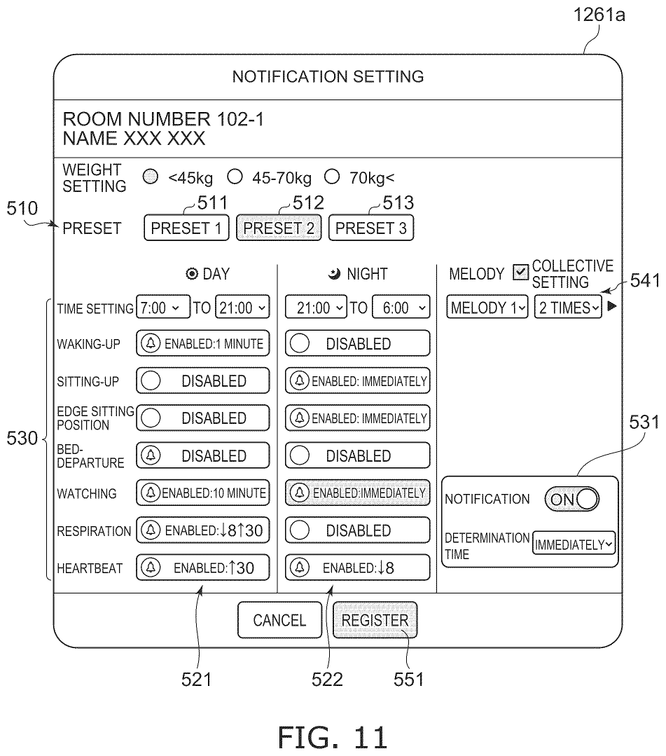

FIG. 11 is a diagram illustrating an example of a notification setting 1261a of the notification setting unit 126. In the present embodiment, the notification setting 1261a is displayed on the PC 51, for example. In the present embodiment, a case where the notification setting 1261a is displayed on the PC 51 will be described, and the notification setting 1261a may be displayed on the smartphone 52 and the tablet terminal 53.

As illustrated in FIG. 11, the room number and the name for identifying the user are displayed at an upper left part in the notification setting 1261a. A setting for distinguishing the body weight of the user is provided on a lower side of the upper left part. In the present embodiment, three sections “<45 kg”, “45-70 kg”, and “70 kg<” are provided. In the illustrated example, the section “<45 kg” is selected.

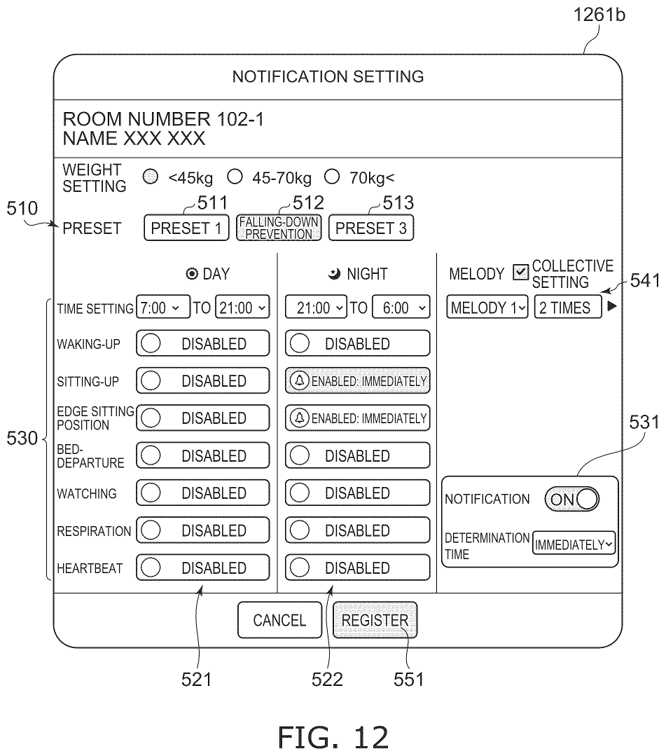

Further, a preset setting unit 510 is provided on a lower side of the three sections. The preset setting unit 510 includes a plurality of preset units 511, 512, and 513. In the present embodiment, the three preset units 511, 512, and 513 are provided. In each of the preset units 511, 512, and 513, different presets are set with respect to the setting of the notification setting in accordance with the states of the user. For each of the preset units 511, 512, and 513, the first notification setting is set for the first time zone 521 in the daytime (day in FIG. 11), and the second notification setting is set for the second time zone 522 in the nighttime (night in FIG. 11). That is, different settings are possible for the daytime and the nighttime. Names of the preset units 511, 512, and 513 can be changed. In an example illustrated in FIG. 12 to be described later, a state where the name of the preset unit 512 is changed from “preset 2” to “falling-down prevention” is shown.

As contents set in the preset units 511, 512, and 513, recommended notification settings are set in accordance with the type of the user, a problem of the user, or the like. As illustrated in FIG. 11, a plurality of notification settings 530 are settings related to waking-up, sitting-up, edge sitting position, bed-departure, watching, respiration, and heartbeat in each of the first time zone 521 in the daytime and the second time zone 522 in the nighttime. The waking-up, the sitting-up, the edge sitting position, the bed-departure, the watching, the respiration, and the heartbeat can be set to be enabled or disabled individually. When the above is set to be enabled, a timing (for example, a time interval, after a predetermined time period, and immediately) at which a notification is performed can be set by a setting unit 531. In addition to these, a setting 541 for a melody can be preset. Specifically, the type of the melody and the number of times the melody is played can be set. The setting of the timing at which the notification is performed and the setting of the melody can be individually performed. When any one of the preset units 511, 512, and 513 is input, the preset setting is reflected and displayed. Here, when a registration button 551 is pressed, a setting in the daytime is registered in the first setting unit 126a, and a setting in the nighttime is registered in the second setting unit 126b. That is, when there is no change from the preset, the notification setting unit 126 registers the settings of the preset as they are as the notification settings in association with the user, and when there is a change from the preset, the content reflecting the change is registered in association with the user.

For example, an initial setting (a preset) when the preset unit 512 is set to the falling-down prevention will be described. FIG. 12 is a diagram illustrating an example when the setting of the preset is “falling-down prevention”. The “falling-down prevention” is a preset presented for a user who is likely to fall down. In this case, all the settings in the daytime are set to be disabled, and a notification of sitting-up in the nighttime is set. In addition, for example, in a case where the preset unit 511 is set to “for a user who often wanders”, in the initial setting, a notification of watching is set in the daytime, and a notification of bed-departure is set in the nighttime. In a case where the preset unit 513 is set to “please pay attention to a change in a physical condition regardless of day and night”, regarding the settings of the respiration and the heartbeat, notification being performed when the number of times of the bed-departure, an upper limit of the respiration (30 times/1 minute), and an upper limit of the heartbeat (110 times/1 minute) are exceeded in both the daytime and the nighttime is set. The present embodiment is not limited thereto, and preset contents of the preset units 511, 512, and 513 can be set in accordance with the states of the user.

The state display program memory 140 stores a state display program. The state display program is a program for acquiring the user information by the detection device 30 and displaying the state of the user to the staff member, an administrator, or the like of the first facility 1a. In the present embodiment, for each user of the first facility 1a, displays of the PC 51, the smartphone 52, and the tablet terminal 53 display, for example, the name of the user, the room number, the position of the detection device 30, the symbol corresponding to the state of the user, the symbol display, the heartbeat rate, the respiratory rate, the time during which the current state continues, the temperature, the humidity, and a bell mark. The contents to be displayed are not limited to the above, but may include other information. Alternatively, a part of the information in the example may not be displayed.

When the state display program is executed, the display setting memory 150 sets to hide or display the information to be displayed among the information indicating the state of the user which is displayed on the display.

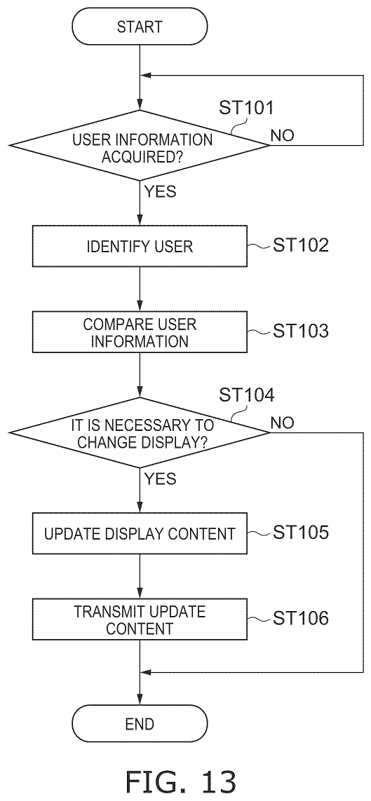

FIG. 13 is a flowchart illustrating an example of the state display program. The state display program is a program for performing a process of displaying the user information and updating a display content each time the user information is acquired. Hereinafter, the process of updating the display content when the user information is displayed will be described.

As illustrated in FIG. 13, the controller 101 determines whether the user information is acquired (ST101). When it is determined that the user information is not acquired (ST101: NO), the controller 101 continues a standby state.

When it is determined that the user information is acquired (ST101: YES), the controller 101 identifies a user based on the acquired user information (ST102). The user information includes the serial number of the detection device 30. Therefore, for example, the controller 101 identifies the user by using the information stored in the personal information memory 122.

Next, the controller 101 compares the acquired user information with the user information stored in the user information memory 125 (ST103), and determines whether a content for changing in display is included (ST104). When it is determined that there is a content to be changed (ST104: YES), the controller 101 updates the display content (ST105). For example, the controller 101 determines whether the symbol display needs to be changed, and when it is determined that the symbol display needs to be changed, the controller 101 changes the outline of the symbol display. For example, when the user in the sleeping state wakes up and turns into the waking-up state, the outline of the symbol display is changed from the circular shape to the hexagonal shape, and the symbol is also changed from the sleeping state ((a-1) of FIG. 8A) to the waking-up state ((b-1) of FIG. 8B). Next, the controller 101 transmits an update content to the system management server 3 (ST106). When it is determined that there is no content to be changed (ST104: NO), the process ends. In this way, the display content is updated in real time. The user information is synchronized between the facility management server 10 and the system management server 3.

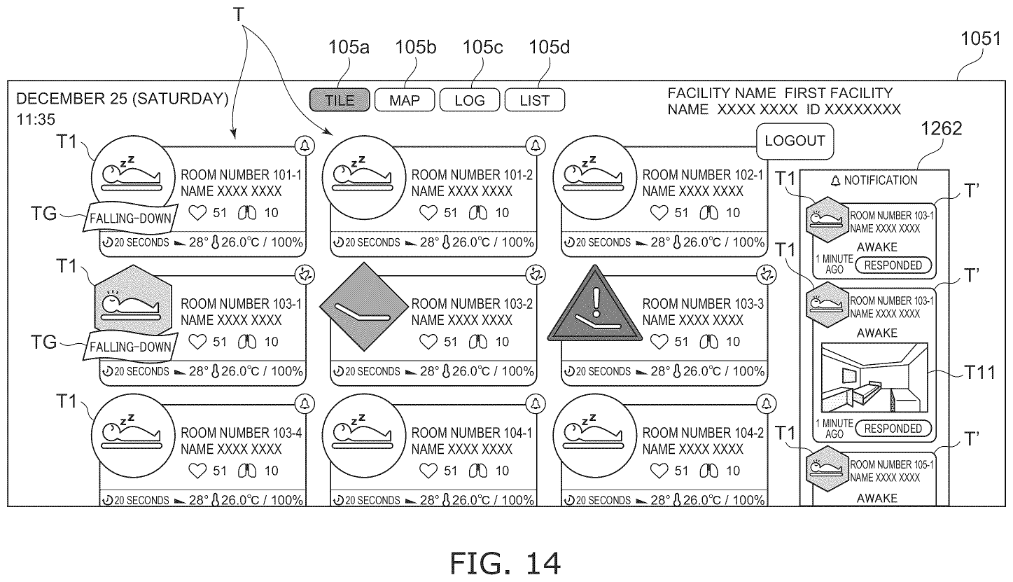

FIG. 14 is a diagram illustrating a display example of the states of the users displayed on the display 105.

As illustrated in FIG. 14, for each user, the state of the user is displayed in a rectangular frame (hereinafter referred to as “tile”) on a display screen 1051. In each of tiles T, a symbol display T1 is displayed at an upper left part. The user information is displayed beside the symbol display T1. Heartbeat rate information per minute and respiratory rate information per minute are displayed below the user information. Further, information on the time during which the current state continues, information on the angle of the back section 21, watching setting information, temperature information, and humidity information are displayed at a lowermost end. Call information, which is a notification based on the notification settings described above, is displayed on an upper right part.

As illustrated in the display screen 1051 of FIG. 14, the outlines of the symbol displays T1 are displayed according to the states of the users. Therefore, the staff member of the first facility 1a can visually recognize, by visually recognizing the displayed circular shapes, that the users for which the circular shapes are displayed are in a safe state at a glance, and can visually recognize a danger level corresponding to the number of angles of the outline of the symbol display at a glance. Specifically, when the staff member glances the PC 51 displaying the states of the users in real time at a station at the time of night shift, and when the staff member checks the states of the users in real time by the smartphone 52 during work, the staff member can easily understand how much each of the users of the first facility 1a is far from the normally assumed sleeping state because the staff member can check it based on any of three ways including the symbol, the outline of the symbol display T1, and the display color of the symbol display T1, and the time required for the staff member to perform a checking operation can be shortened.

There are tiles T in which a tag TG is displayed. The tag TG is displayed on a lower side of the tile T in a distorted substantially rectangular shape. The tag TG may not be displayed on the lower side of the tile T, and may be displayed in the vicinity of the tile T, and more preferably, may be displayed in a manner of partially overlap with the tile T. In addition, an outline of the tag TG is not limited to the illustrated shape and may be other shapes, but it is desirable that the outline of the tag TG is at least different from the outlines of the symbol displays T1. The displaying of the tag TG includes characters such as “BPSD” and “falling-down”. When a notification is performed based on the notification settings of the preset units 511, 512, and 513, the tag TG is displayed according to attributes set in advance for the users. Here, the attribute is set for each type of responses to the states of the user. Furthermore, the attribute is a setting for providing an instruction related to a response to an inexperienced staff member in a simple manner. The staff member can display the users having the same attribute as a list by using the attribute. Examples of the attribute include “a user who requires the staff member to constantly check a change in vital signs”, “a user at a risk of falling-down”, and “a user who has behavioral and psychological symptoms of Dementia (BPSD)”, there users being assumed to require an end-of-life care. When the notification settings using the preset units 511, 512, and 513 are performed with respect to the users in such states, the tags TG are attached to the notification settings. In this way, the tag TG is displayed on the tile T.

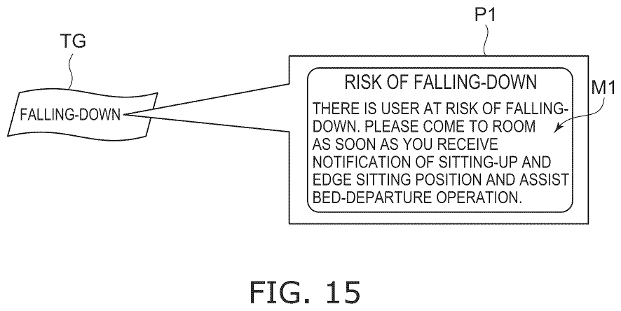

A setting related to the presence or absence of the displaying of the tag TG can be changed by using the user interface device on the display screen 1051, and when the tag TG is clicked or tapped, a content of the response, that is, a care action is displayed. FIG. 15 is a diagram illustrating an example of a pop-up screen P1 displayed when the tag TG (falling-down) is clicked. As illustrated in FIG. 15, in the pop-up screen P1, the “falling-down” and a response method “There is a user at risk of falling-down. Please come to the room as soon as you receive the notification of sitting-up and edge sitting position and assist a bed-departure operation.” are displayed. The content of the response displayed in this manner may be stored in one memory 110 in association with the type of the attribute, for example. The content of the response is stored in advance in a preset, and the content of the response may be changed by a staff member who can use an authority (for example, the organization account) capable of changing the notification setting.

Further, a tile tab 105a, a map tab 105b, a log tab 105c, and a list tab 105d are displayed on an upper central part of the display screen 1051. The tile tab 105a is in a format illustrated in FIG. 15, that is, a format in which for each user, the state of the user is displayed in a tile format. The map tab 105b is in a format in which for each user, the state of the user is displayed in a map format based on the map information described above. The log tab 105c is, for example, in a format in which the information for each user is displayed in units of last two days. The log tab 105c is displayed using the user information for each user stored in the user information memory 125. The list tab 105d is in a format in which the user information for each user is displayed in a list format.

Further, a notification screen 1262 is displayed on a right side of the display screen 1051. On the notification screen 1262, a plurality of notifications to be noticed are displayed based on the setting of the notification setting unit 126. A plurality of notifications T′ are sequentially displayed on the notification screen 1262 in a form similar to the tiles T. For each of the plurality of notifications T′, the room number, the name and the state of the user, and the symbol display T1 are displayed. Further, in each of the plurality of notifications T′, a message indicating the state of the user, a time elapsed after the notification is noticed, and a responded button are displayed. The message indicating the state of the user is, for example, “awake”. In this way, messages corresponding to the waking-up state, the sitting-up state, the edge sitting position state and the like of the user are displayed. The responded button is pressed by the staff member when the response to the notification from the user is performed. When the responded button is pressed, the corresponding notification T′ is deleted from the notification screen 1262. Here, as an example of the response to the notification, for example, the staff member comes to the room of the user and checks the state of the user. When the notification screen 1262 is displayed, the melody set by the notification setting 1261a may be played.

For example, the plurality of notifications T′ of the notification screen 1262 are arranged in a notification order, and the staff member operates the user interface device to scroll the screen, and thus it is possible to display the immediately previous notification screen or the immediately subsequent notification screen. In addition, when a button 401 is pressed, or when the notification is not deleted after 10 minutes from the occurrence of the notification, for example, the notification is deleted from the notification screen 1262.

When one of the tiles T is clicked, a detail screen for displaying the state of the user in more detail is displayed. Hereinafter, a detail screen 1262a will be described in detail.

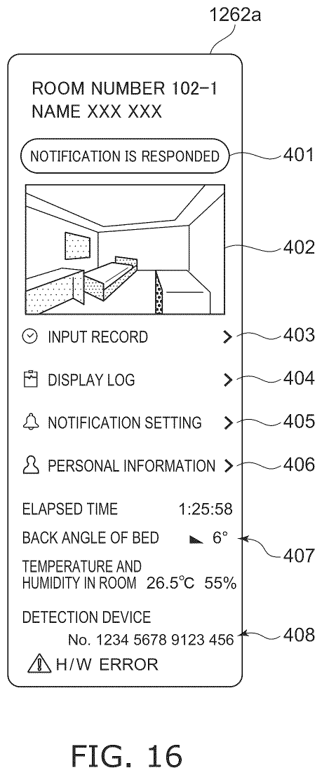

FIG. 16 is a diagram illustrating an example of the detail screen 1262a.

As illustrated in FIG. 16, the detail screen 1262a includes the button 401, a screen 402, an input instruction unit 403, a display instruction unit 404, a notification setting instruction unit 405, a personal information instruction unit 406, an environment display 407, and a serial number 408 of the detection device 30.

The button 401 is a button for inputting the fact that “the notification is responded”. That is, the button 401 is a button to be pressed when the staff member responds the notification displayed on the detail screen 1262a. The button 401 is a button for providing the same instruction as the responded button described in the notification screen 1262. In a predetermined time period, the number of notifications T′ displayed on the notification screen 1262 is the number of notifications, and the number of notifications for which the button 401 (and the responded button in the notification screen 1262) is pressed among the notifications is the number of responded notifications which are responded by the staff member. When the notifications are generated, the user, an ID of the staff member who presses the responded button, and the presence or absence of the pressing of the responded button are also recorded in association with each of the notifications. The screen 402 reflects a situation of the room of the user. For example, when a camera is installed to capture an image of the user in the motorized bed 20 or the room R, and the image of the camera can be acquired, the image captured by the camera is displayed on the screen 402. When the camera is not installed, for example, the screen 402 may not be displayed.

When the staff member provides care or the like, a necessary recording item is input to the input instruction unit 403. When the input instruction unit 403 is clicked, a screen for inputting a work record is displayed. The staff member inputs, on the displayed screen, the presence or absence of visits, an excretion related item (wearing of a diaper, use of a toilet, an amount of urine, a state of a stool), a postural change (a left lateral position, a spine position, and a right lateral position), and the like. The above items are associated with the user and the ID of the staff member as the care record, and are recorded in, for example, the personal information memory 122. When it is desired to display the log related to the user, the display instruction unit 404 is pressed. When it is desired to change the notification setting, the notification setting instruction unit 405 is pressed. When it is desired to display the information stored in the personal information memory 122 as the personal information or the information acquirable via the network 2, the personal information instruction unit 406 is pressed.

The environment display 407 displays the time elapsed since the state becomes the current state (the waking-up state, the edge sitting position state, or the like), a back angle, and the temperature and the humidity in the room. These pieces of information may be acquired, for example, by using the serial number of the detection device 30 used by the user at the time of notification.

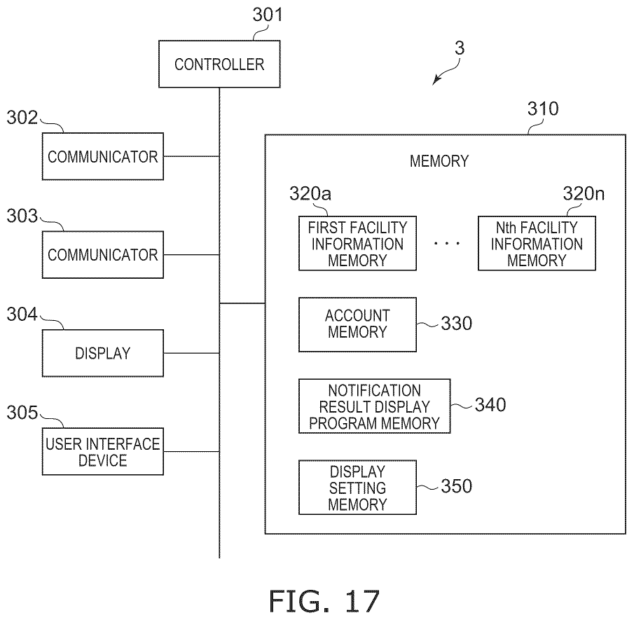

FIG. 17 is a diagram illustrating an example of a control configuration of the system management server 3.

As illustrated in FIG. 17, the system management server 3 includes the controller 301, the communicator 302, a communicator 303, a display 304, a user interface device 305, and a memory 310. The memory 310 includes a first facility information memory 320a to an Nth facility information memory 320n, an account memory 330, a notification result display program memory 340, and a display setting memory 350. The display setting memory 350 stores a setting of information to be displayed on the display 304, a threshold, and the like.

The controller 301 controls the components in the system management server 3. The communicator 302 is an interface that transmits and receives information to and from the facility management servers 10 in the first to Nth facilities via the network 2. The communicator 303 is an interface for transmitting and receiving information to and from the tablet terminal and the smartphone. The display 304 is, for example, a liquid crystal display. The display 304 displays information to an operator who operates the system management server 3. In the present embodiment, for example, the states of the users of the first facility 1a to the Nth facility 1n are displayed in real time. The user interface device 305 is a keyboard, a mouse, or the like. With respect to the information displayed on the display 304, the operator operates the user interface device 305 to give a predetermined instruction.

The first facility information memory 320a to the Nth facility information memory 320n store information stored in the first facility information memory 120 to an Nth facility information memory, respectively. For example, as described in FIG. 7, the first facility information memory 320a stores the same contents as the information stored in the map information memory 121, the personal information memory 122, the symbol memory 123, the symbol display memory 124, the user information memory 125, and the notification setting unit 126. The synchronization of these pieces of information is executed in real time. When the symbol memory 123 and the symbol display memory 124 are common to the first facility 1a to the Nth facility 1n, the symbol memory 123 and the symbol display memory 124 may not be included in the first facility information memory 320a to the Nth facility information memory 320n. In the present embodiment, a case where the information is stored in synchronization between the facility management servers 10 of the first facility 1a to the Nth facility 1n and the system management server 3 is described, and the present embodiment is not limited thereto. The plurality of pieces of information on the first facility to the Nth facility may be stored only in the facility management servers 10 of the first to Nth facilities, and the system management server 3 may display the plurality of pieces of information stored in the facility management servers 10. The plurality of pieces of information on the first facility to the Nth facility may be stored only in the system management server 3, and the facility management servers 10 may display the plurality of pieces of information stored in the system management server 3.

The account memory 330 stores an account for limiting a function of displaying a state of a user, a function of displaying a care record or the like, and a function of changing various settings (for example, the notification settings). As described above, similar to the facility management server 10, the four accounts are stored. When a user of the system management server 3 logs in by using the organization account, the system management server 3 can display the states of the users in all facilities of the first facility 1a to the Nth facility 1n.

The notification result display program memory 340 stores a notification result display program. The notification result display program is a program for displaying, for each of the first facility 1a to the Nth facility 1n, or for each predetermined unit (any method for creating the unit such as a group for a facility, a floor number of a facility can be used), notification result information such as the number of persons executing the notification settings in the daytime or in the nighttime, the number of notifications per predetermined time unit, and the number of notifications not responded. Since the number of responded notifications can be acquired by the inputting of the button 401, the number of notifications not responded can be obtained by subtracting the number of responded notifications from the number of notifications. In the present embodiment, the number of notifications and the number of notifications not responded are displayed in a graph. When the user logs in by using the facility account described above, the notification result display program is executed to display a notification result based on the notification result information of the corresponding facility, and when the user logs in by using the organization account, the notification result program is executed to display notification results based on the plurality of pieces of notification result information of all facilities.

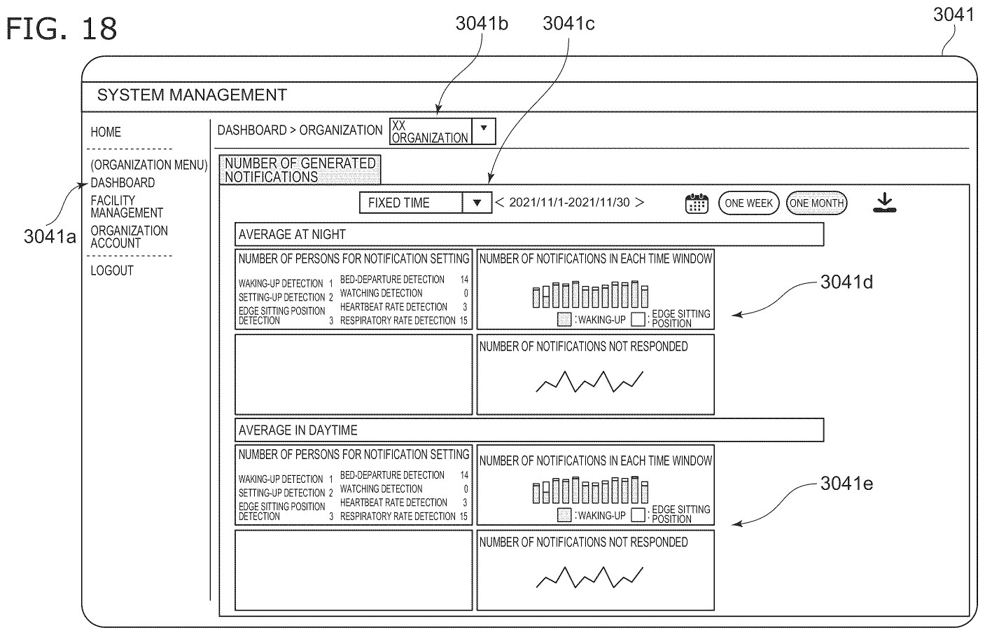

FIG. 18 is a diagram illustrating an example of a dashboard screen 3041 on which a notification result is displayed. The dashboard screen 3041 is displayed when a dashboard button 3041a is input from a menu displayed on a left side of the dashboard screen 3041.

As illustrated in FIG. 18, a button 3041b and a button 3041c are displayed on the dashboard screen 3041. The button 3041b can designate a unit for displaying a notification result, such as an organization, a facility, a group, a unit, or the like. In the illustrated example, xx organization is selected. The button 3041c can select a period during which the notification result is output. In the illustrated example, 2021 Nov. 1 to 2021 Nov. 30 (November, 2021) is selected as a fixed period. The period can be set freely.

In addition, a notification result in the nighttime 3041d and a notification result in the daytime 3041e are displayed. For each of the notification result in the nighttime 3041d and the notification result in the daytime 3041e, the number of persons for notification setting (the number of persons set for each of the waking-up detection, a sitting-up detection, an edge sitting position detection, an bed-departure detection, the watching detection, a heartbeat rate detection, and a respiratory rate detection) is displayed, and the number of notifications in each time window is displayed as the notification result. The number of notifications in each time window is, for example, the number of notifications every hour, and is indicated by a bar graph. Lower parts of portions in the bar graph indicate the number of notifications of the waking-up detection, and upper parts of the portions in the bar graph indicate the number of notifications of the edge sitting position detection. On a lower side of the bar graph, a polygonal line graph of the number of notifications not responded is displayed. This polygonal line graph is based on a unit corresponding to the bar graph (every hour in the present embodiment). Since the bar graph and the polygonal line graph are arranged along a vertical direction, the operator of the dashboard screen 3041 can visually recognize the comparison between the number of notifications and the number of notifications not responded easily.

Next, a function of proposing notification optimization will be described.

First, a case where the notification optimization is performed by reducing the number of notifications will be described.

When a ratio of the number of notifications in a certain time window to the number of users of the facility is equal to or larger than a predetermined threshold, it is displayed that there may be too many notifications. Here, the predetermined threshold is defined according to the type of the notification content. For this display, for example, when the dashboard screen 3041 is displayed, a pop-up display indicating a proposal for changing due to the large number of notifications is performed. When the pop-up display is clicked, a specific content of an optimization proposal of reducing the number of notifications is displayed.

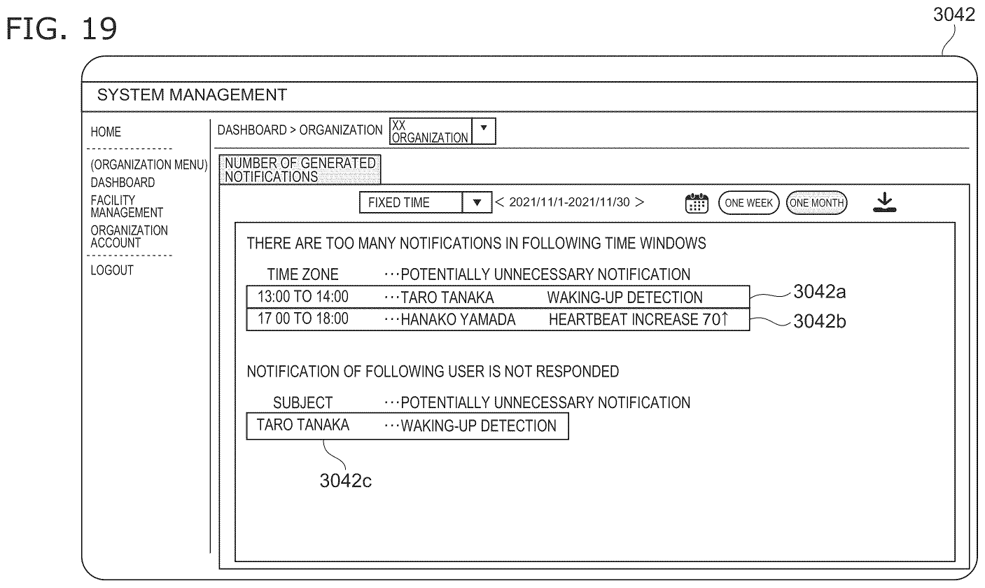

FIG. 19 is a diagram illustrating an example of a dashboard screen 3042 that displays a proposal content.

As illustrated in FIG. 19, the time window, the name, and the notification content are displayed on a lower side of a message “there are too many notifications in the following time window”. In the illustrated example, “the number of notifications for the waking-up detection of Taro Tanaka is equal to or more than a predetermined number in a time window of 13:00 to 14:00” is displayed on the display 3042a, and “the number of notifications for heartbeat rate increase 70 (notifications that are made when the heartbeat rate per minute exceeds 70) of Hanako Yamada is equal to or more than a predetermined number in a time window of 17:00 to 18:00” is displayed on a display 3042b. On a lower side of the display 3042a and the display 3042b, a message “the notification of the following user is not responded” is further displayed. “Taro Tanaka, waking-up detection” is displayed on a display 3042c as “subject . . . potentially unnecessary notification”. As described above, a proposal related to a notification that is potentially unnecessary among a plurality of notifications is displayed. That is, it is determined based on the following conditions. A first condition as to whether the notification is an unnecessary notification is that there are notifications the number of which is equal to or larger than a predetermined number in a certain time window, as illustrated in the display 3042a and the display 3042b. A second condition as to whether the notification is an unnecessary notification is that the number of notifications not responded is larger than a predetermined number, as illustrated in the display 3042c. In the illustrated example, both of the notification satisfying the first condition and the notification satisfying the second condition are displayed, and when only one of these conditions is satisfied, only the notification satisfying this condition is displayed. In the present embodiment, the determination is made for each of the first condition and the second condition, and when both the first condition and the second condition are satisfied, a notification may be displayed as a proposal of reducing the number of notifications.

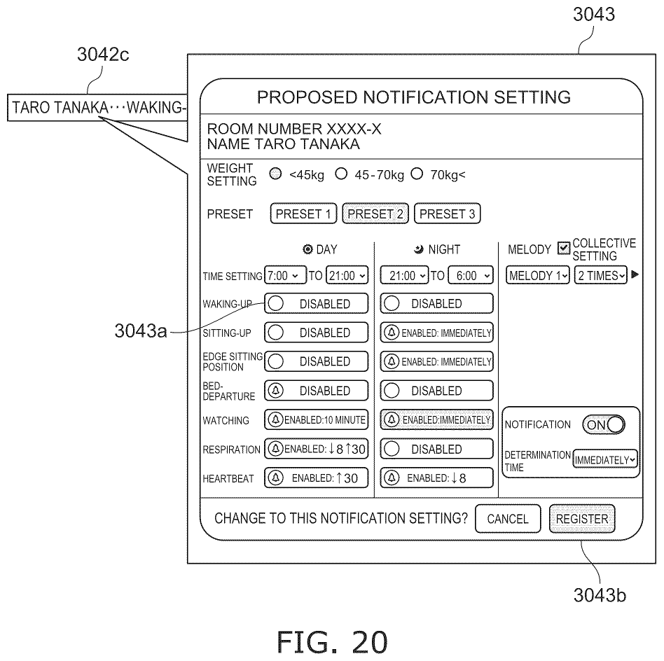

In the dashboard screen 3042, when the display 3042a, the display 3042b, and the display 3042c are clicked, a pop-up display 3043 is displayed. FIG. 20 is a diagram illustrating an example of a pop-up display when the display 3042c is clicked. As illustrated in FIG. 20, the same display as the notification setting 1261a described above is performed. The pop-up display 3043 is different in that the pop-up display 3043 clearly shows it is a proposal, such as “proposed notification setting”, and as illustrated in a display 3043a, the notification setting of the waking-up in the daytime is set to be disabled. In this way, in response to the fact that the number of notifications for the waking-up detection of Taro Tanaka in the time window of 13:00 to 14:00 is large, it is proposed that the notification of the waking-up detection of Taro Tanaka in the daytime is set to be disabled. Further, it may be proposed to change the time setting, and since there are many waking-up detections from 13:00 to 14:00, it may be proposed to change the time setting in the daytime to 7:00 to 13:00. Here, when the operator presses a registration button 3043b, a content of the proposed notification setting is overwritten and stored in the notification setting. In the present embodiment, a notification for the waking-up of Taro Tanaka in the daytime is set to be disabled by the notification setting unit 126. In this way, it is possible to reduce the number of notifications.

In addition, the number of staff members working in the facility is different between daytime and nighttime. Specifically, the number of staff members working in the nighttime is smaller. For this reason, it is necessary for the staff member to pay more attention to the users of the facility as compared with the case in the daytime, and the threshold in the nighttime that defines whether the number of notifications is large is also lower than that in the daytime. The operator operates the user interface device 305 to display the dashboard screen 3041 that displays the proposed notification setting described above. Regarding the dashboard screen 3041, for example, the dashboard screen 3041 is displayed when a manager managing work operations examines improvement of the work operations, and the manager examines a change in the notification settings based on the display content. On the dashboard screen 3041, the controller 301 may display a pop-up screen that provides a proposal of disabling the notification setting for the user that has sent too many notifications or that has sent a notification for which the response has not been completed. In this case, when the notification content is an increase or decrease in the heartbeat rate or when the notification content is an increase or decrease in the respiratory rate, the pop-up screen may provide a proposal that the thresholds of the heartbeat rate and the respiratory rate are changed to be reduced, while the thresholds of the heartbeat rate and the respiratory rate are kept as they are. For example, in a case where the number of notifications is large such as when the number of detections of the increase in the heartbeat rate is 70 or more for one minute, the threshold is set such that the notification is enabled when the number of detections is 80 or more for one minute. In this way, the number of notifications may be reduced.

Further, even in a case where the number of notifications does not exceed the threshold, for a user who is determined based on statistics for each type of notification and each user that a ratio of his/her notifications which have required a longer time until responded completely to all notifications or a ratio of his/her notifications not responded to all notifications is equal to or larger than a certain value, the controller 301 may provide a proposal of setting the notification setting of the corresponding user to be disabled.

By reducing the number of notification settings in this manner, it is possible to detect a case “although the notification setting is set, no response is actually required even if the melody of the notification is played”, and it is possible to set an appropriate notification setting by providing a proposal of setting a notification for this case to be disabled.

Next, a case where the notification optimization is performed by increasing the number of notifications will be described.

In the present embodiment, a help button “when there is a problem in setting” is provided on the screen displaying the notification settings.

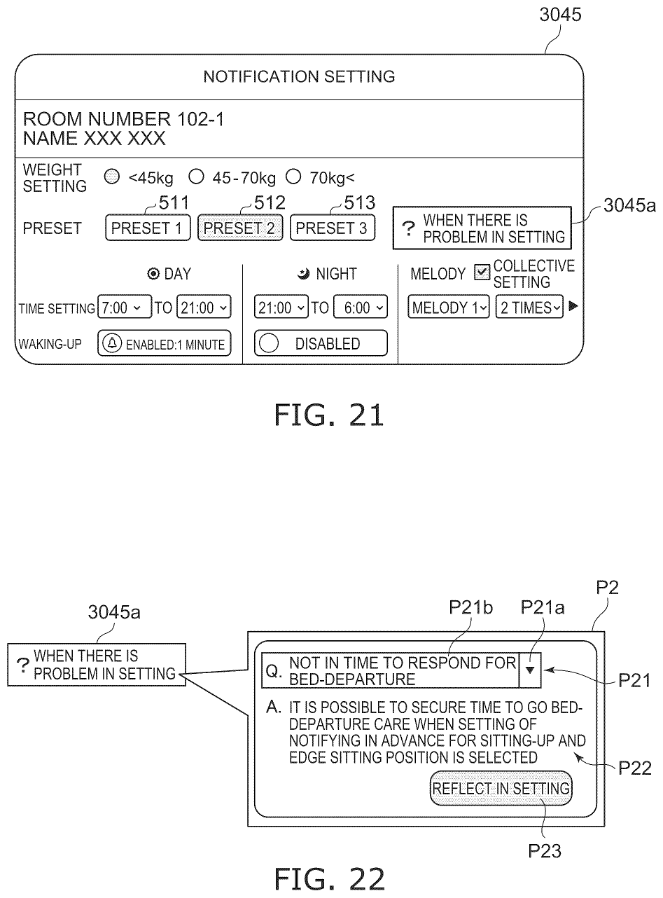

FIG. 21 is a diagram illustrating an example of a dashboard screen 3045 in which the button “when there is a problem in setting” is displayed for the notification setting described with reference to FIG. 11.

As illustrated in FIG. 21, a “when there is a problem in setting” button 3045a is displayed in the vicinity of the preset setting unit 510. In a case where the “when there is a problem in setting” button 3045a is selected, a setting for increasing the number of notifications is proposed in accordance with the selection.

FIG. 22 is a diagram illustrating an example of a pop-up screen to be displayed when the “when there is a problem in setting” button 3045a is selected.

As illustrated in FIG. 22, in a pop-up screen P2, a display P21 indicating a content of a question, a display P22 indicating an answer to the question, and a button P23 for reflecting in a setting are displayed. The display P21 is of a pull-down type, and when a button P21a is pressed, a message content of a message unit P21b changes. In FIG. 22, a question “Q. not in time to respond for bed-departure” is displayed. As an answer to the question, a message “A. It is possible to secure a time to go bed-departure care when a setting of notifying in advance for sitting-up and edge sitting position is selected” is displayed on the display P22. In a case where it is desired to set this content, the button P23 is pressed, and the content is set by the notification setting unit 126. That is, only the notification setting is overwritten to the current notification setting and set by the notification setting unit 126.

By increasing the notification setting in this manner, it is possible to perform an appropriate notification setting.

### Second Embodiment

In the present embodiment, a case of setting a display range of a notification set by the notification setting unit 126 will be described. The same components as those of the first embodiment are denoted by the same reference numerals, and detailed description thereof will be omitted.

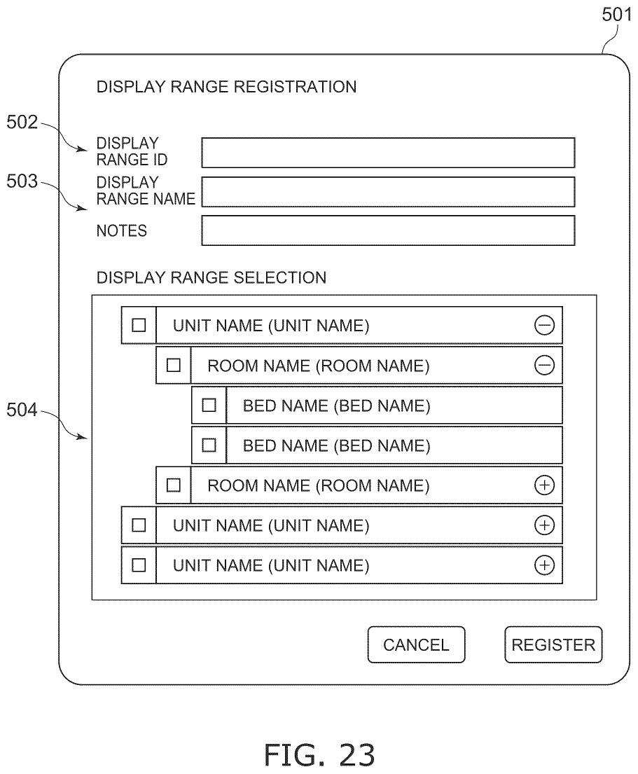

FIG. 23 is a diagram illustrating an example of a display range setting screen 501. The display range setting screen 501 is displayed on the display of the information processor, and is used for a case where a staff member or the like sets and edits a display range.

As illustrated in FIG. 23, a display range ID unit 502, a display range name unit 503, and a display range selection unit 504 are displayed on the display range setting screen 501. The display range ID unit 502 displays an ID assigned at the time of new registration. When editing is performed, the display range ID unit 502 is used to identify a display range to be edited. The display range name unit 503 displays a name set to facilitate finding of the display range set by the staff member. Therefore, staff members of various types can easily select the name as the display range.

The display range selection unit 504 is a display for selecting a display range. In the present embodiment, the display range selection unit 504 is a hierarchical structure including a unit name, a room name, and a bed name. The unit name identifies a unit, the room name identifies a room, and the bed name identifies a bed. The bed may be identified by the detection device 30 instead of the bed name. Similarly to the first embodiment, the unit is a group for a facility, a floor number of a facility, or the like. Each of the unit name, the room name, and the bed name is provided with a check box at a front portion thereof. By checking the check box, the display range in the corresponding unit is set. For example, if the unit is checked, the states of the users included in the unit are set as a display range. In this way, since the display range can be set hierarchically in a manner such as the unit name, the room name, and the bed name, it is possible to finely set the display range. For example, the display range can be set such that a staff member receives a notification from Mr. XX only although the room is out of an assigned range thereof, and it is also possible to cope with a case where an assigned range of a staff member is irregular. A case of new registration or editing is possible, for example, only when a user logs in with an authorized account, such as the organization account. For example, regarding registration and editing of a display range name to be described later, the display range name can be set and edited only on a registration screen displayed when the user logs in with the organization account.

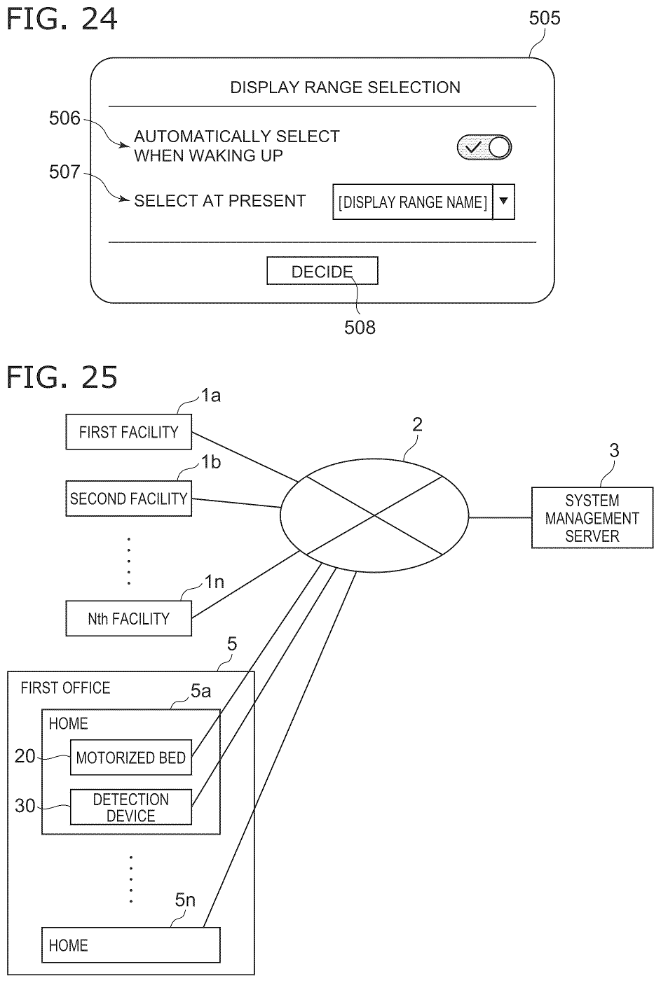

FIG. 24 is a diagram illustrating an example of a display range selection screen 505. For example, when the staff member starts up the state display program and logs in, the display range selection screen 505 is first displayed. The display range selection screen 505 also can be displayed from a system setting menu.

As illustrated in FIG. 24, a display 506, a display 507, and a decision button 508 are displayed on the display range selection screen 505. The staff member sets the display range by the display range selection screen 505. Therefore, it is possible to set the display range for each information processor. When the user logs in for the first time, or when the setting of the display 506 is OFF, the display range selection screen 505 is displayed. Whether to automatically select the display range at start-up is set by the display 506. For example, in a case where one tablet terminal is used by only one staff member, when the display 506 is set to ON in advance and the staff member next logs in to the tablet terminal, the setting of the display range set when the staff member logged in the last time can be inherited, and the screen for setting the display range can be skipped. In addition, for example, in a case where one tablet terminal is used by a plurality of staff members, when the display 506 is set to OFF in advance, the display range can be selected at the next login. The display 507 displays the display range name of the currently selected display range. When the decision button 508 is pressed, the notification is displayed in real time in the display range set in the display range name input to the display 507. As a result, for example, only the notification from a user who is managed by the staff member using the smartphone 52 or the tablet terminal 53 can be displayed on the notification screen 1262.

### Third Embodiment

In the embodiments described above, a case where the system management server 3 displays the states of the users using the plurality of facilities 1a to 1n as illustrated in FIG. 1 is described, and the embodiments are not limited thereto.

FIG. 25 is a diagram illustrating an example of the entire network configuration.

As illustrated in FIG. 25, in addition to the configuration illustrated in FIG. 1, the motorized beds 20 and the detection devices 30 of users using a service provided by a first office are communicably connected to the network 2. The first office is, for example, an office that provides a home care service. For example, when a first service provider is registered in the first facility 1a, information on a user at home (a names of the user at home, a serial number of the motorized bed 20, and a serial number of the detection device 30) is stored in the facility management server 10 in association with a registration number of the first office. The user information detected by the detection devices 30 (which may include the information detected by the motorized beds 20) can be displayed in real time on the facility management server 10 via the network 2, and the registration information and the settings can be changed. The information on the users at home may be stored in the system management server 3 instead of the facility management server 10, and the user information of the users may be transmitted to the system management server 3. With such a configuration, it is possible to display the states of the users using the detection device at home without using the facility.

In the above description, the embodiments have been described with reference to the specific examples. However, the embodiments are not limited to the specific examples. For example, a person skilled in the art may similarly practice the embodiment by appropriately selecting specific configurations of components such as the sensors in the detection device, the processor, the processing circuit, the movable part, and sections from known art, and such practice falls within in the scope of the embodiment to the extent that similar effects can be obtained.

A combination of any two or more components of the specific examples within a technically feasible range falls within the scope of the embodiment to the combination includes the extent that the spirit of the embodiment.

In addition, all of notification optimization systems and management servers practicable by an appropriate design modification by a person skilled in the art based on the notification optimization systems and the management servers described above as the embodiments also fall within the scope of the embodiment to the extent that the modification includes the spirit of the embodiment.

In addition, in the category of idea of the embodiment, various modifications and corrections can be conceived by a person skilled in the art, and these modifications and corrections are understood to be embraced within the scope of the embodiment.

The embodiments include the following aspects.

### APPENDIX 1

A notification optimization system comprising:
a first setting unit to which a first notification setting in a first time zone including noon is set, the first notification setting being selected from a plurality of notification settings;
a second setting unit to which a second notification setting in a second time zone including twelve midnight is set, the second notification setting being selected from the plurality of notification settings; and
a controller configured to perform a notification based on settings of the first setting unit or the second setting unit.

### APPENDIX 2

The notification optimization system according to appendix 1, wherein
the first time zone and the second time zone do not overlap each other.

### APPENDIX 3

The notification optimization system according to appendix 1 or 2, wherein
the plurality of notification settings are different with respect to a state of a user detected by a detection device,
the notification optimization system further includes a preset setting unit including a plurality of preset units for presetting the different settings, and
the settings of the first setting unit and the second setting unit are set based on settings of the preset units.

### APPENDIX 4

The notification optimization system according to any of appendix 1 to 3, further comprising:

a display configured to display a state of the user for each user, wherein
when the first setting unit and the second setting unit are set based on the settings of the preset units, in addition to the display of the state of the user, the display displays a tag that is displayed according to an attribute set by the user.

### APPENDIX 5

The notification optimization system according to appendix 4, wherein
the attribute is set for each type of responses to a state of the user.

### APPENDIX 6

The notification optimization system according to appendix 5, wherein
when the tag is input, the display further displays a response content corresponding to the attribute.

### APPENDIX 7

The notification optimization system according to any of appendix 3 to 6, wherein
the display displays a help button in a vicinity of the preset setting unit,
when the help button is displayed, the display displays a help content, and
when the help content is input, the first setting unit and the second setting unit reflect the input help content in the first notification setting and the second notification setting, respectively.

### APPENDIX 8

The notification optimization system according to any of appendix 1 to 7, further comprising:

a memory configured to store whether a response to a content notified by the controller is performed.

### APPENDIX 9

A system management server comprising:
a communicator configured to acquire, from a plurality of the notification optimization systems according to appendix 8, notification result information including the number of performed notifications and the number of notifications not responded, and
a display configured to display the notification result information.

### APPENDIX 10

The system management server according to any of appendix 9, wherein
when the number of notifications within a predetermined time period is equal to or larger than a predetermined number based on the notification result information, the display displays that the number of notifications is equal to or larger than the predetermined number.

### APPENDIX 11

The system management server according to appendix 10, wherein
the predetermined number is changed in the first time zone and the second time zone.

### APPENDIX 12

The system management server according to appendix 10 or 11, wherein
the display displays a proposal to change a notification setting for which the number of notifications is determined to be equal to or larger than the predetermined number into a setting of not performing the notification, or a proposal to change the setting into a content of reducing the number of notifications.

### APPENDIX 13

The system management server according to appendix 12, wherein
even in a case where the number of notifications does not reach the predetermined number within the predetermined time period, when statistics of contents of the notifications indicate that a ratio of notifications, which have required a longer time until responded completely than a predetermined time, to all notifications or a ratio of notifications not responded to all notifications is equal to or larger than a predetermined ratio, the display displays a proposal to change the notification setting into the setting of not performing the notification, or a proposal to change a content of the setting into the content of reducing the number of notifications.

1. A notification optimization system comprising:
a display;
a memory configured to store data of notification setting; and
a processor configured to:
cause a first graphical user interface for notification setting to be displayed on the display, the first graphical user interface including one or more selectable objects to select one or more preset notification settings, each of the one or more preset notification settings corresponding to a specific physical condition of a notification subject;
perform, based on a user operation on one of the one or more selectable objects on the first graphical user interface, a first notification setting in a first time period including noon, the first notification setting including a plurality of notification settings for a plurality of physical states of the notification subject on a bed, respectively;
cause, based on the user operation on the one of the one or more selectable objects on the first graphical user interface, the first graphical user interface to display a first plurality of notification setting objects indicating the plurality of notification settings of the first notification setting in the first time period, respectively;
perform, based on the user operation on the one of the one or more selectable objects on the first graphical user interface, a second notification setting in a second time period including midnight, the second notification setting including the plurality of notification settings, the second notification setting being different from the first notification setting;
cause, based on the user operation on the one of the one or more selectable objects on the first graphical user interface, the first graphical user interface to display a second plurality of notification setting objects indicating the plurality of notification settings of the second notification setting in the second time period, respectively; and
perform a notification based on the first notification setting and the second notification setting.

2. The notification optimization system according to claim 1, wherein
the first time period and the second time period do not overlap each other.

3. The notification optimization system according to claim 1, wherein
the plurality of physical states of the notification subject includes a first physical state detected by a detection device and a second physical state detected by the detection device,
the first physical state being a wake-up state in which the notification subject is awake lying on the bed and the second physical state being a sitting-up state in which the notification subject is sitting up on the bed.

4. The notification optimization system according to claim 3,
wherein
the processor is configured to cause a second graphical user interface for showing a current physical state of the notification subject to be displayed on the display,
when the first notification setting and the second notification setting are performed, the second graphical user interface includes:
an illustration indicating the current physical state of the notification subject in a shape associated with the current physical state; and
a tag associated with the unique physical condition corresponding to the selected preset notification setting.

5. (canceled)

6. The notification optimization system according to claim 4, wherein
in response to a user selection of the tag, the display further displays a response content corresponding to the unique physical condition corresponding to the selected preset notification setting.

7. The notification optimization system according to claim 3, wherein
the first graphical user interface includes a help button in a vicinity of the one or more selectable objects to select one or more preset notification settings,
when the help button is selected, the display displays a help content and a second selectable object, and
when the second selectable object is selected, the first notification setting and the second notification setting are performed to reflect notification setting corresponding to the help content.

8. The notification optimization system according to claim 1, wherein
the processor is further configured to store in the memory whether a response to the notification has been performed.

9. A system management server comprising:
a communicator configured to acquire, from a plurality of the notification optimization systems according to claim 8, notification result information including the number of performed notifications and the number of unanswered notifications for which there was no response; and
a second display configured to display the notification result information.

10. The system management server according to claim 9, wherein
when the number of performed notifications within a predetermined time period is equal to or larger than a predetermined number based on the notification result information, the second display displays that the number of performed notifications is equal to or larger than the predetermined number.

11. The system management server according to claim 10, wherein
the predetermined number is different between the first time period and the second time period.

12. The system management server according to claim 10, wherein
the second display displays a proposal to change the first or second notification setting for which the number of performed notifications is determined to be equal to or larger than the predetermined number into a notification setting with which a notification is disabled, or a proposal to change the first or second notification setting into a notification setting that produces fewer notifications under the same circumstances.

13. The system management server according to claim 12, wherein
the notification result information further includes the number of tardy notifications for which there was a response outside a predetermined amount of time, and
even in a case where the number of notifications does not reach the predetermined number within the predetermined time period, when a ratio of tardy notifications to the performed notifications or a ratio of unanswered notifications to the performed notifications is at least a predetermined ratio, the second display displays one or more of the proposals.
